(19) United States
(12) Patent Application Publication
(10) Pub. No.: US 2007/0285758 A1
(43) Pub. Date: Dec. 13, 2007

(54) Medical apparatus with light flux for biological treatments

(76) Inventors: D. Ion Cristian Degeratu, Ilfov (RO); V. Vladimir Constantinescu, Bucharest (RO)

Correspondence Address: HERSHKOVITZ & ASSOCIATES, 2845 DUKE STREET, ALEXANDRIA, VA, 22314, US

(21) Appl. No.: 10/587,209

(22) PCT Filed: Jun. 21, 2004
(86) PCT No.: PCT/RO04/00010

§ 371 Date: Jul. 25, 2006

(30) Foreign Application Priority Data
Jan. 27, 2004 (RO) A 2004 00069

(51) Int. Cl. A61N 5/06

(52) U.S. Cl. 359/235; 600/476

(57) ABSTRACT

The invention refers to a medical apparatus with light flux for biological treatments, which uses the effects of an electromagnetic radiation pencil for obtaining modifications at atomic level, which can be used for treating and ameliorating some diseases. The apparatus is designed for degenerative energetic-diseases, specific to our living in the nowadays society: vices, stress, feeding, sedentary living; but for some genetic heredity aspects, too. The electromagnetic radiation pencil is processed by an incandescence bulb (4) set in the focus of a parabolic mirror (6) and transmitted to a shutter disc (8) equipped with a slits regulating device (9), run by an engine (12). A digital modulator of the light flux (13) directs the shutter disc. The light pencil is focused in an objective (10), equipped with an optic filter (11), which has to be modulated at much lower frequencies than the used radiation (longer wave lengths) for obtaining a deeper distance in penetrating the tissue. A computer (16) controls the apparatus' handling and coordinating. The medical apparatus doesn't use electromagnetic fields that are injurious to our health, it permits the secure obtaining of the necessary frequencies needed in treating some diseases and it's easy to use.

The invention refers to a medical apparatus that uses the effects of the electromagnetic field on living organisms, using an artificial flux of well-determined color for obtaining some modifications at atomic level that can be used to cure or meliorate some diseases or to relax and refresh the body.

The apparatus is designed for curing energetic-degenerative diseases specific to our living in nowadays society, vices, stress, feeding, sedentary living, heredity or genetic deficiency.

About 100 years ago, the Danish doctor NIELS RYBERG FINSEN laid the foundation of modern light therapy.

The Nobel Prize for medicine was awarded to FINSEN in 1903 for his activity in light therapy. He was the one who designed the first apparatus that technically generated beams similar to solar ones.

There are certain advantages in using the solar beams artificially generated such as: the parameters of intensity and the spectrum of the generated light are controllably so they are reproducible.

At about the end of the 1960's the German Neuropathologist PETER MANDEL rediscovered the light therapy.

He said that every color of the spectrum is in strong connection with body's health: “the color penetrates the body' cells carrying with it the needed information for curing”

After trying this unconventional therapy JACK ALANACH, a telecommunication' specialist studied the phenomenon and reached the following conclusion: “Light contents the anti-virus program exactly like anti-virus programs for our computers”.

Those who practice colorpuncture use the KIRLIAN technique for determining the diagnosis. The KIRLIAN photos detect the energetic field of the body and so detect the energetic perturbations.

But long before PETER MANDEL, this idea about light therapy sprang up in the mind of a young man—CAROL PRZYBILLA. Born in Romania (1902), CAROL graduated from two faculties in Germany where he met and became a friend of ALBERT EINSTEIN.

In fact EINSTEIN himself was the one who awoke his interest for studying the light. Even from that time he had the idea of designing an apparatus that could generate “solar light”.

Something is certainly true: he started a new chapter in light therapy, a very little known domain, but one that carried out, studied and experimented could be a very notably one. Using the information that we have now from OSIM Romania (State Office for Inventions and Marks) we can consider the fact that more than 40 inventions of this type—using light—conform with the enclosed table (table 1) are registered in the whole world. These inventions are divided into three types:

I. Laser Type Therapy

Inaugurated in 1967, it is characterized of using a certain wavelength and an energy transmitter, which is incorporated. Laser light is polar (vibrates in one single plan) and can be amplified at very high energies.

Even from that time it has been established that this type of light has a bio-stimulator effect on cells. So it means that tissues can be vaporized, we can effectuate very precise cuts and many other things depending on the laser type that we use. New possibilities of using different types of laser are published every year.

II. BIOPTRON Type Therapy

The spectrum of infrared and ultraviolet rays was the one that was used in modern light therapy at the beginning of the photo-medicine development.

In 1981 a team of Hungarian research-workers created a source of light that was initially based on laser therapy, of generating a combination of visible light and infrared radiation. The team considered light polarization as being an important parameter. The therapy system type BIOPTRON was created on this technology basis.

It is the most recent achievement that is used at international level right now, together with laser type therapy, having very good results and being also very well sold at a large scale.

It uses polychrome polarized light having wavelength and different colors, which has specific effects on cells. Polarization means that magnetic waves of light vibrate in parallel plans.

Built in many different types (different powers) it performs a superficial treatment on bodies especially at epidermis level.

a) Compact BIOPTRON

It is designed to be used at home, but also for special treatment in professional domains, too.

The apparatus consists of a special reflector with halogen bulb (20W) and a fan. It has a timer inside which emits a tone at an interval of 2 min. It has to be delivered together with oxy-spray 250 ml.

b) BIOPTRON II

It is designed for professional use and it corresponds to international standards TEC-601-1. Bioptron II lamp is easy to handle. The case made of polyuretanical foam contents the whole optics of a reflector with halogen bulb (100 W) and the timer and the electronic section, too. The treatment duration can be selected from 1 to 15 min. Zepter Company, one of medical BIOPTRON AG orientation in Romania, has patented two certificates of invention:
a) Therapeutic lamp having registration no. 96-00803/14.08.1995
   international claim no. EP 95/03220/14.08.1995
   international publication WO 96/04958/22.02.1996
b) Therapeutic lamp irradiating polarized light, designed for manual use, having claim no. 96-00804/14.08.1995
   international publication no. WO 96/049508/22.02.1996

In the project for apparatus' description, BIOPTRON AG ZEPTER Company settled the method that makes possible the following activities:
to influence the biologic system in a positive and commode manner
to activate the capacity of self-defense
to activate regenerating processes

These apparatuses emit polarized light, which is emitted linearly on wavelengths between 400-9000 nm.

The light waves cover the treated area on the visible side and the infrared spectrum. In this way cells can be treated till the depth of 2.5 cm under the skin layer, without a significant heating of the tissues.

They do not contain ultraviolet rays and they have energy of 40 nv/cm².

There haven't been notified any secondary effects in using this apparatus so far. Because the wave spectrum is not genetically absorbed there can not be later genetically defects.

The treatment has the following contra-indications and it is not recommended for:
treating cancer
   pregnant women
   persons with pace-marker

III. Heliostroboscop Type Therapy

Conform with the international patent WO 92/13597(20.08.1992) having no practical applicability, and left at experimental level.

Our present invention is labeled under the following title: “MEDICAL APPARATUS WITH LIGHT FLUX”, it belongs to HELIOSTROBOSCOP type and the present version has PROBIOFOTON title. It is designed as an original version, based on an electromagnetic field, emitted with a source of light, and it also has the purpose of making this way of treatment known at therapeutic level,

The present invention's purpose is to spread the application of biological treatments of irradiation with a light flux of determined-color.

The medical apparatus for biological treatments with a light flux it is used for modulating, separating and focusing a pencil of electromagnetic radiation emitted by a bulb with incandescence for obtaining energetic modifications in cellular biochemistry.

The medical apparatus resolves the problem of treating the non-congenital diseases with the aid of some bright radiation.

The technical problem that our present invention solves is to avoid the use of electric and magnetic fields in the light flux processing.

The medical apparatus, according to the invention, resolves the above problem because the electromagnetic field applied to a sick person it is obtained from a source of light put in the focus of a parabolic mirror.

In the light pencil's way, processed as we mentioned above, a rotating shutter and on objective are to be assembled on one of the case's inner sides, objective on which an optic filter is set.

A computer interface helps commanding and operating with the use of a program that controls the entire work of the apparatus.

The shutter disk has two elliptical orifices, which as a result of the variable-regulating device, design an ellipse with a little opening, less at one end, and circle-shaped at the end of the second ellipse.

The bright flux this way obtained is composed of direct rays from the light source and rays reflected from the parabolic mirror, focused through a lens.

The final pencil of rays this way obtained, after following through this optic labyrinth, has to be modulated at much lower frequencies comparing to the radiation that was used (higher wave lengths) for obtaining a long distance in penetrating the tissue.

The medical apparatus, according to the invention, has the following advantages:

a) assures the certain obtaining of the necessary frequencies needed in treating some diseases

b) now it is possible to use the treatment for people who have a pace-marker, and this is for the first time in applying this type of treatment using light

c) this type of therapy can not only be combined with other types of treatment but can also fulfill their effect

d) allows the co-ordination, depending on the case, of the naturist methods based on body detoxification and on introducing in diet some certain vitamins depending on the type of disease

e) a very wide area of disease can be cured using it

f) rapid and lasting results are obtained

The apparatus (1st picture), according to the invention, is made of a case 1 in which on a base plate 2 it is fixed a support 3 of the incandescent light type bulb 4.

For ensuring uniformity of ventilation for the bright item—bulb type 4, a fan 5 will be set in front of a main orifice a.

The interference between the incidental ray and the reflected ray, in coherent conditions, is obtained through one bulb operating.

The type of bulb 4 that we use—an incandescent one, has to be a pencil of light rays focused as much as possible on one point. The pencil's power is given by:
the capacity of transmitting to distance
the ray intensity
the possibility of mechanic modulation, for avoiding the overheating effect on skin.

The power of bulb 4 with incandescence depends on the type of tissue that we irradiate. A gradual treatment of the sick person is recommended, proportional with the intensity of his/her disease at that time, till using the maximum intensity of the bulb 1500W, depending on case.

The source of light, bulb 4 type is set in the focus of a parabolic mirror 6 made of a material with a high absorption coefficient for some frequency categories.

The mirror 6 positioning has to carry out the parallelism condition between the incidental ray and the reflected ray, having the possibility of displacing through the handle bar b.

The bulb 4 positioning to the reflecting item, mirror 6, has to carry out the conditions of obtaining some parallel light pencils.

For positioning the bulb 4 regarding the efficiency of the apparatus, in accordance with the gradual use of the its power, a gradual system 7 of bulb operating is recommended.

In the light pencil's way, processed as we mentioned above, a rotating shutter 8 is to be assembled on one of case's inner sides. This rotating shutter has two elliptical orifices c d (1^{st} figure) which using a regulatory device for slits 9 (2^{nd} figure) design other different forms more efficient in treating.

The forms and dimensions c d on the rotating shutter 8 become variable depending on the frequency of the photons flux.

The shutter 8 opening slits with variable geometry figure the form of a small ellipse c at one end and the form of a small circle d at the other.

The ray processed on the shutter disc goes through an orifice e made on the inner side of the case and it is then directed through a lens 10 on which an optical filter 11 is fixed.

The shutter disk 8 is operated by an engine 12, which is directed by a digital voltage regulator 13 for obtaining the needed modulation frequencies (3^{rd} figure).

A fan 14 ventilates both the engine 12 and the digital voltage regulator.

The description and the scheme of the digital voltage regulator 13 in 64 stage displaying the engine's step 15 are the following:

It is a complex montage at the first sight having a relatively big number of chips, but at a closer sight you can see that every chip alone, is ordinary used, conforming to the dated in catalogue. Let's start with dividing the scheme in operating blocks:

A) Displaying block

B) Digital-analogic counter and decoding block

C) Command and control block

D) Power source

The first functioning block consists of two counters (MMC 40192), at whose outlets are connected to 7 segment/ach decoder/driver (MMC 4543), followed by two digits with common anode.

The second block consists of two counters (MMC 40193) from which the first six bits are selected, commanding six relays through a buffer with open-collector outlets. On the relays contacts there are parallel-assembled resistances, which during the countering make a variable resistance, depending on the pitch number, which varies the gate current of a triac through a diac.

Command and control block is made of the following circuits: MMC 4013 dual D-TYPE FLIP-FLOP; MMC 4048 (the logical programmable gate with eight inputs); MMC4060 (14 STAGE RIPPLE CARRY BINARY COUNTER/DIVIDER AND OSCILLATOR); two MMC circuits 4093 (four SI-NU logical gates (trigger Schmidt). The first bistable in MMC4013 chip receives the command “emergency stop” on the first ‘reset’ through a positive impulse from a reversible switch. The second command set on the same bistable is linked to the pin “clock” and execute the start-stop function. The bistable is linked in configuration of divider by two, it means that “data” pin is linked at the negative outlet. The outlets in fact represent the start/stop and inhibition physical commands, which are visually observed on a bicolor led, commanded by two bipolar transistors. When the led is red it means the montage is in “stand-by”, when in fact the counters are initialized (MMC 40192 and MMC 40193) on the “preset enable” pin, charging the registers with the binary code 60. The meter of the divisor MMC 4060 is to be reset, and the MMC 4543 decoding-systems receive a command to turn the digits off and through a relay the engine's power is cut off. When a positive impulse shows up on the “clock” pin, the bistable moves back, the led will light green, and the MMC 40192 and MMC 40193 meters will receive the incrementation from the binary preset number, which is 60. Concomitantly, the MMC 4060 divisor will start to generate the clock signal for the above mentioned meters. The MMC 4543 diver will be enable to light the digits and the relay will be charged letting the voltage to power the engine. The next bistable of the MMC 4013 circuit controls the numbering direction, letting the clock signal (processed from MMC 4060) through two logical gates SI-NU, go to the incrementation or decrementation pins of the MMC 40192 and MMC 40193 meters. The “sef” pin will receive a positive impulse through a condenser, from the “true” outlet of the other bistable, which will validate incrementaion when starting. Where “data” pin is, there is also the outlet of a gate SI-NU with two entries, which set the next numbering direction - depending on the “true” outlet of the bistable on the first entry, and on the second entry linked at the significant bit among those six that command the relays. This data will be charged when a positive impulse will appear at the “clock” pin, processed in the control block made of a MMC 4048 and a MMC 4093. This impulse appears in the moment when on the entries of the logical gate MMC 4048 there is the binary code “0” or “63”, which means “000000” or “111111” that represent the end of the descremenation or incremenation, resulting from those six outlet bits of the MMC 40193 circuit. In any of those two situations, at the outlet “J” of the MMC 4048 gate, the logical “0” level appears. During incrementation, this gate will have the logical function SI-NU, and during decrementation—the logical function SAU-NU. Everything will be controlled at “Ka” pin linked to true outlet of the control bistable of direction. The outlet “J” of the logical gate MMC 4048 commands an astable with auto-reverse made of the four logical gates SI-NU having two entries each, of the second MMC 4093. When the 0 level appears in the entrance of the first gate, considering the fact that on the second gate is fixed the first logical level, the outlet would be in the logical level 1. The second gate receives on one gate the same fix level (logical 1) and n the other one the logical level 1 resulted from the first gate, the result on the outlet being the change into logical zero. There is a condenser between the outlet of the second gate and one of the entrances of the third one. This condenser is charged through two resistances 220 k and 390 k, through which it would discharge itself, when the logical level 0 appears from the outlet of the second gate. Until the condenser' discharging, the third gate will have at the outlet 0 logical level. The impulse will be inverted by the fourth gate, which has its entrances connected together, and transmitted to the “clock” pin of the control bistable of the clocking direction. This one will charge the logical level in “data” changing or not the metering direction. The last chip MMC 4060 produces the clocking signal visually indicated by the decimal point of the digit, which displays the decimal fractions. The oscillator frequency is varied by a potentiometer, so the signal received at the Q4 outlet will have it's own variable frequency, too. The metering signal could be a manual one (from a reversible switch) or an automatic one coming from the above-mentioned circuit, a commutable by a two position switch.

The power source is as simple as possible, made of: a transformer, a rectifying bridge, a filtering condenser and two stabilizing integrators of 5V and 12V, the first one powers the C-MOS circuits, it means the logical part; and the second charging the relays. Both circuits use the same heather sink.

This montage is integrated in the main functioning scheme of this apparatus, of transmitting a modulated light flux. In this way, the modulated flux once filtered through the shutter disk it is modulated in 64 degrees with the help of this digital modulator, for obtaining variable frequencies of the photons emitted by the source of light.

The flux modulation, initially done with the purpose of growing the penetrating distance into the treated tissue, represents an element of novelty versus former technical achievements.

In conclusion, using this electronic device, the penetration distance into the tissue was possible to become deeper than 12 cm, a great performance in comparison with the former results.

A computer 16 controls the apparatus' handling and coordinating.

For an efficient ventilation cycle, there were designed two ventilation slits (f g) in the back side of the a apparatus, concordant with the ventilation made by (5-14) fans, both in the superior side and near the foot plate.

The final pencil resulted after going through this optic labyrinth has to have a minimum intensity, to be mechanic modulated at much lower frequencies comparing with the used radiation (longer wavelengths), for obtaining o deeper distance of penetration into the tissue.

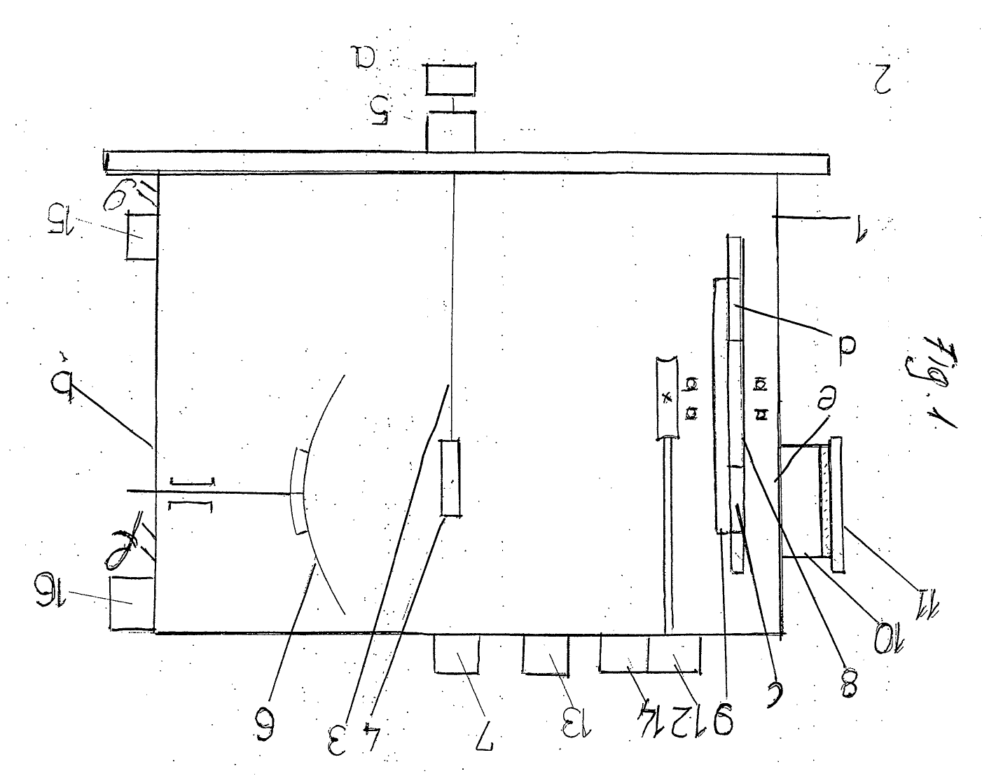

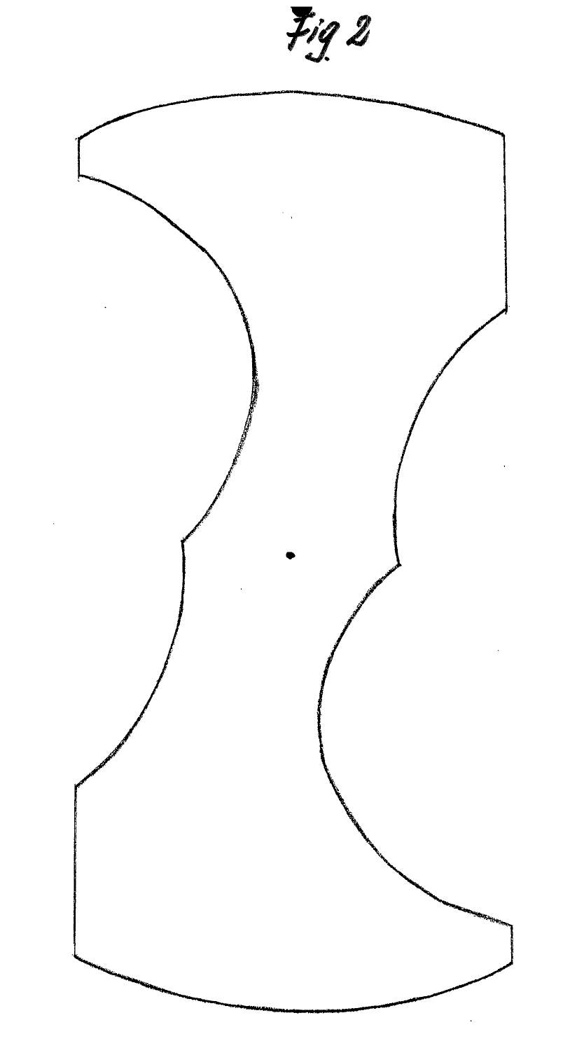

Reviewing the constitutive elements of this MEDICAL APPARATUS WITH LIGHT PENCIL (FIG. 1) we remind the following:

I. Main Elements

1) case

2) foot plate

3) bulb support

4) incandescent bulb

5) bulb fan

6) parabolic mirror

7) gradual system for lightning the bulb

8) coring shutter

9) slits regulating device

10) apparatus' object

11) optic filter

12) rotating disc's engine

13) digital modulator of the light flux

14) engine's fan

15) engine's pitch display

16) computer

II. Additional Accessories

a) main orifice in ventilation

b) handle bar for the mirror

c) shutter disc' orifice with slit for handling

d) objective orifice

e) case's ventilating slit

III. Other Different Constituting Elements

FIG. 1 The ensemble scheme of the apparatus

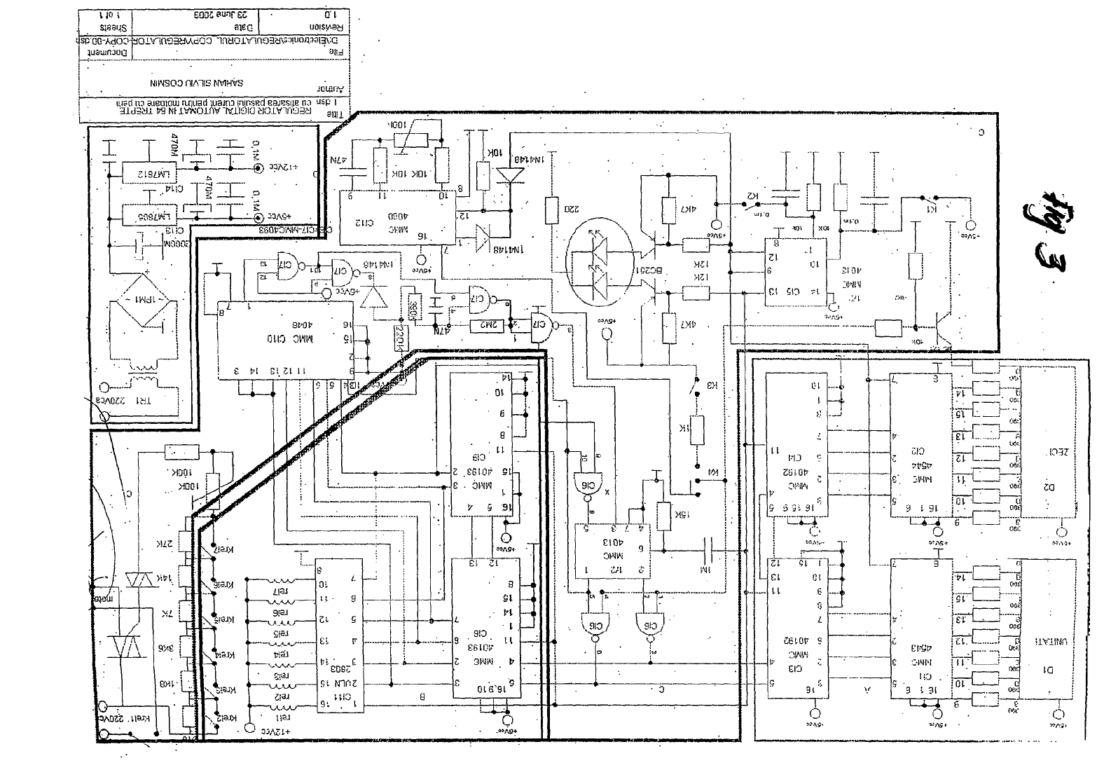

FIG. 2 Slits regulating device

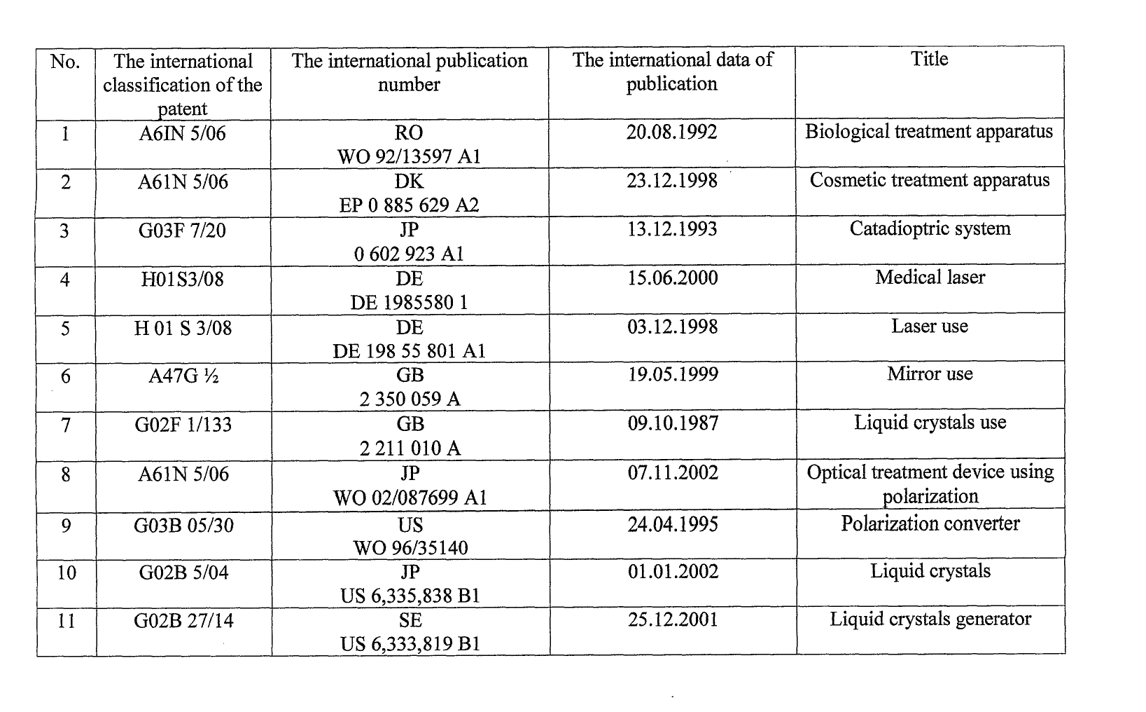

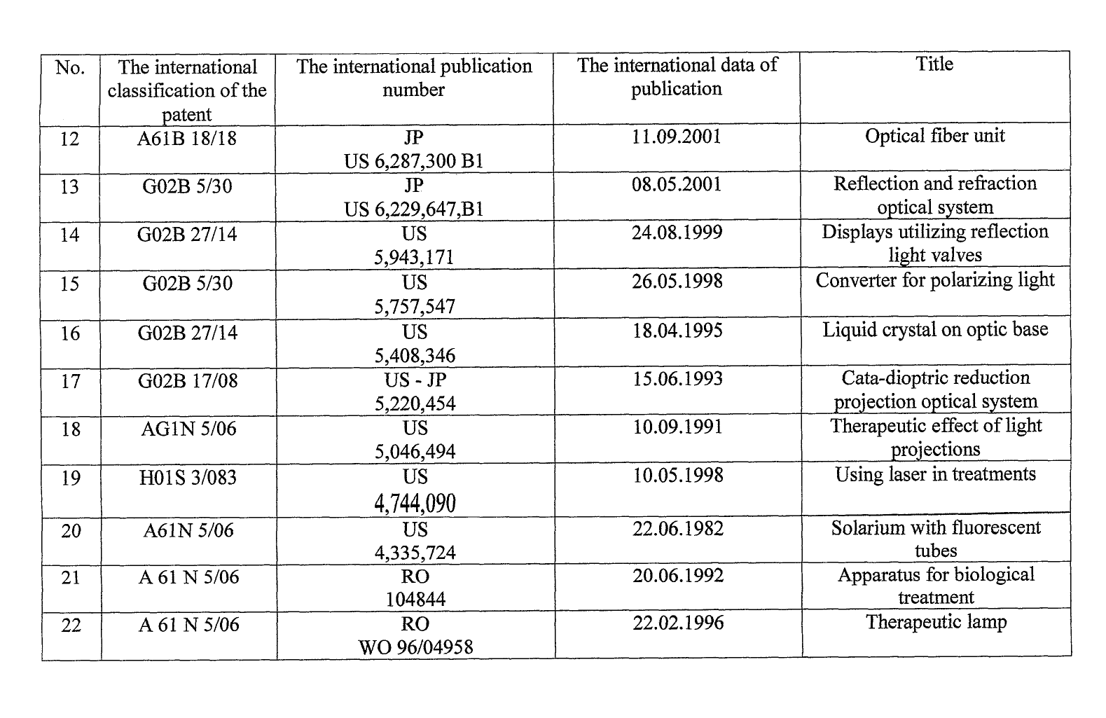

FIG. 3 The electronic scheme of the light flux' digital modulator

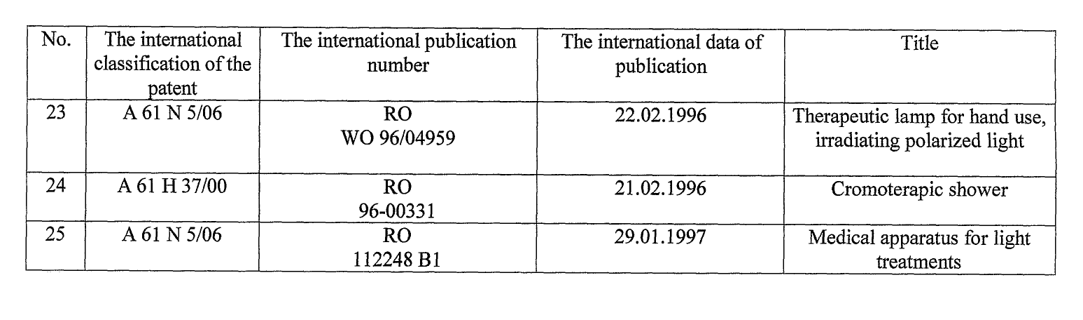

FIG. 4 Elliptical slits' disc

IV. Table of Inventions that use the Light Flux in Therapy

(annex IV)

V. Excerpts from Patents that use Light in Therapy

(annex V)

The most representative inventions at international level, which are opposed to our invention are patented as follows:

1. PATENT WO 0921/13597-20.08.1992 ROMANIA
inventor CAROL PRZYBILLA

2. PATENT WO 96/04958-22.02.1996
solicitant BIOPTRON AG-MONCHALTOR F (CH)

The elements that make us different from all these are:

I. The Shutter Disc 8 Performances

As a result of our studies, we ascertained that the shutter disc dimension of 150 mm, according to patent WO92/1397(1992), should be changed to the optimum dimension of 220 mm.

The shutter disk 8 dimension it's proportional with the light flux intensity and concords with the rotating frequency of the disc. Using this way calculated shutter disc 8, the frequency modulation of the electromagnetic field is possible, for deepening the penetration distance into the tissue.

II. Regulating Slits Device 9

Associated with the dimension of the shutter disc 8, makes the two ellipses have different forms and dimensions, in contrast with WO 0921/1357. If in this patent, the ratio of the ellipses' axis of the orifices (cd) is 066-095, in our description for patenting, the form and the dimension of cd slits on the shutter disc 8 are variable, depending on the photons' flux frequencies, which are imposed by the sick people' diagnosis.

Due to the slits regulating device 9, the orifice of the slit c designs the form of a small ellipsis, and the orifice of the slit d designs the form of a small circle.

In WO 092/13597 patent, the slits which are very big have the disadvantage of a weaker control for obtaining the modulation frequencies.

The new system proposed for patenting has the advantage of penetration frequencies in the tissue, so ensures a growth in the efficiency of this treatment, which is an absolute novelty for this type of medical apparatus with light flux.

III. The Digital Modulator of the Light Flux 13

After many years of studying we have reached the conclusion that also the system of varying the frequencies with the help of the digital modulator 13, with the current pitch of the engine display 15, is concordant with the energetic centers of different types of tissue treating, subdue to cells' modifications.

The rotating frequency of the shutter disc 8 is variable for encountering the area of necessary requirements in treating the energetic centers of many different types of tissue.

The area of speed for the shutter disc is between 1550-3000 turn/min., opposed to WO 092/13597 patent where the frequency variation is inexistent.

Both the obtaining of the variation of frequencies and that of a certain types of photons in the electromagnetic field that is used, doesn't depend on the nurse's medical training in using the apparatus, since it is an automatic system.

IV. The Bulb Power (4) Supplier of the Electromagnetic Field

Higher from 600-1000W in WO 092/13597 patent, to 1500W.

It is another important item that makes us different from all the others opposable patents mentioned above.

The length of the irradiating flux at this level it's another innovation which makes the described energetic treatment more efficient.

It is enough to remind about the Bioptron type apparatus used today, where the maximum power of the light when functioning can not be over 100W.

V. Electromagnetic frequency band used when referring to this type of apparatus is between 560-3000 nm, a frequency obtained through the variation of the functioning parameters of the computer's command and regulation systems.

The variation of the electromagnetic frequencies can be also obtained if varying the power of the incandescent bulb, being electronically controlled and the power area being set in the computer's command program.

VI. The Engine's Pitch Display

It is the most interesting innovation because allows us a permanent control of the apparatus' functioning parameters when doing the treatment.

VII. Computer's (16) Help in Coordination

Through its functioning, the computer assures the command and control for all variable items inside the system, the ability of the stuff that supervises it being not necessary for this activity.

VIII. Turning Cylinders (12) in WO 092/13597 Patent

They were eliminated because they were practically inefficient for obtaining a beating phenomenon during the modulation and interference process of the light pencil.

IX. The apparatus dimension is obvious superior to those two types of opposable apparatuses described above, and it is imposed by:
the shutter disc 8 dimension
the engine 12 placement
the incandescent bulb's power 14
the parabolic mirror 6 dimensions
the ventilation and fanning system (5-14 f, g)
computer's placement 16

While the opposable patents were made for an ambulatory treatment—being portable, the functioning of this apparatus and the method of treatment require a special room, because its power can lead to other diseases or adverse reactions, that need a closer look from the doctors.

X. The non-noxious treatment is valid if the technically conceived parameters and their functioning are respected.

Due to the certain wavelengths that we use, we can cure sick persons who:
bear a pace-markers
bear metallic rods

Due to our experience, sick persons that could follow this treatment could be between 1-80 years old; the optimum results being obtained when the ideal age is between -60 years old if the human cell is receptive to the modifications imposed by the apparatus.

XI. Suggesting a sound theory verified during a 12-year period of private experience, both on animal and human tests. We subject to appreciation one excerpt of this theory in this patent of invention and debate.

XII. The parabolic mirror (6), made of a material with great absorption coefficient of some frequencies' categories is specially designed.

XIII. This is a complementary treatment to allopathic and naturist medicine that offers a substantial support in obtaining the best results.

XIV. A new methodology in sick people's treatment, a very efficient one which was strictly verified during the time and also totally different versus the one that has been lately used in light treatments. Depending on the types of diseases we can predict symptoms and reactions, so that the sick person being treated can verify the treatment efficiency and its synchronization with the complementary associated methods.

It is known that among the four interactions existing in the nature, only the unionized electromagnetic one has the power conditions and the long range needed to govern and influence the biologic processes of the living world. The ionized electromagnetic radiation (including the ultraviolet one) has destructive effects on the biologic world, reducing every experience to violent non-controlling mutations. It's about the energies' area of the electromagnetic field, having an essential role in reactions that involve the recognition of the complementary structures, as for example: the rejoining, the transcription and the repair of DNA and the translation of the genetic message in the protein's biosynthesis, too

It can be theoretically and experimental demonstrated that certain physical laws, through the energy of the electromagnetic field lead all mechanisms that stock and operate with biologic information. The purpose is—knowing that any affection represents a genetic deficiency at a certain level of the organism—to cure. The basic cause, it is the introducing and fixing at informational level, of inadequate atoms from the energetic point of view, because of an inadequate feeding in most of the cases. At the DNA's level, the apparition of these atoms for a long period of time destabilizes the weaker chemical links that are directly responsible for maintaining the genetic information. Specially, it is about the inter-chaining links among the four nucleotides, that at the chemical level represents the hydrogen bridge. These links' disconnection for too long periods of time, on certain sequences of the DNA's molecule, except for the natural mitosis or meiosis phenomena that are controlled by the body, leads to informational modifications at some certain genes' level, to the synthesis of some unspecific amino-acids—proteins. So, all these lead to the cellular metabolism' affection. This fact leads further to the non-integration of these new-formed cells in the metabolic interrelations of the entire biologic complex, so, to the disease's appearance.

The curing attempts, at chemical level through medicines, have mostly only the role of correcting the causes' effects of the disease's appearance, the genetic information modification. That's why, basically, these treatments lead most of the times to an improvement, a delay of the disease' evolution. More than that, through the processing procedures, synthesis' processed medicines introduce in the body chemical substances, which used for a long period of time, lead to fixing in our body atoms with inadequate superior energies.

If we try, and this is possible only at physical level, with energetically modifications on the atomic levels of the DNA molecule, we can directly cancel the cause of the disease's appearance, so the rectification of the genetic information. This is possible at the quantum physics' level.

Theoretically we found out that the energies corresponding to the hydrogen's links fixing are energetically compatible with atomic transitions corresponding to a limited number of wave length in the electromagnetic spectrum. The transitions resulted as following the interactions are subjected through certain values to the incertitude principles Heisenberg (the equation between time and energy), that's why experimental measurements are impossible. Theoretically, we can find the field of values with the aid of some wave functions corresponding in Schrodinger's equation.

Through bombing with photons (of above-mentioned energies), it appears when the existing levels' split, at the level of the quantum number m₁, which corresponds, to the necessary energies for recovering the nucleotide's sequences, corresponding to the genetic informational local standard of the specie. Only the magnetic component of the electromagnetic field is responsible for these needed changes, the electric one having an insignificant effect at thermal-dynamic level.

One of the positive immediate aspects, but not a main one is the change of the magnetic properties. It means that the paramagnetic susceptibility is to be stressed through eliminating diamagnetic substances with an energetic destabilizing role. Among all these, water and free hydrogen have important roles, being considered chemical elements with strong pathological effect when accumulated in the cell, knowing that nitrate basis are strongly hydrophobic.

According to the present theory, primitive biological systems (virus, bacterium, and malign cell) operate informational at energetic superior levels, with short life cycles, high mitosis and multiple spontaneous reactions. It means that a primitive life system, for example a virus, would attack a specialized superior one, through energetic modifications at distance, using the electromagnetic field at a quantum level. The energetic modification finally induces informational modifications in the attacked body, being coordinated and controlled by the perturbing system. That's why a conversion ENERGY-INFORMATION appears, where through energetic modifications, informational modifications are produced. So we can afford the hypotheses: an energy quantum—an information quantum. Pathology comes from the intelligence degradation and biological differences. If the evolution's way was long and difficult, the opposite one is much more faster because of the existence in every cell, of the genetic ancestral element of evolution.

For superior organisms, the central nervous system is the leading organ that keeps the energetic order and the cytospecialization to all levels. It means that in the every people's whole life, destabilizing elements induce energies at different specialized cell' levels. The brain has the role of an energy extinguisher. It creates potential differences between itself and the rest of the body, and using the electronic flows it takes over at different levels, the supplementary cellular energy that destroys the specialization. If this does not happen, the cells tend to degenerate to a common standard, which affects the general functioning (e g malign cells). The intelligence is present in every cell, but it has to be stressed through strict energetic properties.

It means that the brain behaves like a charger that charges itself chemically and has, among many others, the role of ENERGY-INFORMATION converting, not only through its intelligence but also using the local existent biological intelligence.

But things are not as simply as they look like, otherwise the pattern would be too simple and could not work at the existence complexity level. The second control system of the entire biological level it's firstly done to the decision block—hypothalamus, and secondly to the entire endocrine system, which through specialized subroutine performs energetic corrections, informational ones.

This control system equilibrates the possible informational dysfunction at different cellular systems, the purpose being to pass through different metabolic equilibrium states, so the entire system protection. When this one can not keep the control of these perturbations any more, it is said that the biological phenomena become critic, the chaos appears which leads to death.

As a conclusion, we may say that every external agent which varies the energy at atomic level, in an increasing mood inside the cell, affects the genetic information through the biological phenomenon of ENERGY-INFORMATION conversion.

So, if we all can say that no matter what the damaging agent is (virus, bacteria, toxicity a s o) the disease means the destruction of the cellular specialization through biological conversion type ENERGY-INFORMATION, let us see what the opposite process—of curing—will be like.

We can use for this, the electromagnetic interaction for energetic modifications in a very strict sense, in accordance with the operating pattern of biological systems. This consists in the possibility of emphasizing magnetic properties on atomic systems.

For changing atomic magnetic properties in biochemical reactions, we can interfere with an external electromagnetic field. The electric oscillation' structure of the interaction's mediator, photon, is not a significant one, it only creates a caloric static macro-effect at the level of the biological system in ensemble.

What we are interested in is the magnetic structure of the electromagnetic field. The external magnetic field interacts with the orbital magnetic moment of he electrons at the level of the m quantum number. There are atoms that have the orbital magnetic moment opposed to the exterior field orientation, so they are rejected out of it, and they are said to have diamagnetic properties. For other atomic systems, the orbits align to the exterior field and this one attracts them, and we say that they are paramagnetic susceptible.

As every kind of substance has in certain conditions diamagnetic properties that vary with temperature and mass, we can use this argument for supporting our experience. In other words we can control the isotopic concentration directly in the living body. It means that we can produce energetic conversions, informational ones, directly on the molecules that stock DNA and operate with genetic information (all types of cellular RNA). The interaction period is 10⁻¹⁷-10⁻¹⁹ seconds, much less than biochemical reactions' speed, so that the biological system should not perceive the intervention.

The biological intelligence is based on atoms and molecules, not on their macro-energetic systems.

Now, back to our problem, in order that the success be assured, it is necessary that these energetic conversions be directed firstly on tissues and organs that have the role of transmitting and regulating of the information in the biologic body. Conform to this theory the curing is performed by the body, and the success is assured, being the only one capable of something like that.

The interventions must be sensible and well measured, and a relaxing period is required between them, for emphasizing and reappearing of the biological intelligence characteristic to the system we work on. Such a treatment is artificially stimulated but the cure is natural, processed by the affected system

With these energetic modifications apparently insignificant, a body can start the cure against cancer. All the energetically affected cells, so informational affected till a certain energetic limit, will eliminate cancer, conforming to omni-potential /multi-potential theory. Those that are irreversibly affected, including the malign ones, will be found and attacked by the immunity system, for being cleared. The procedure is a long one because our method can not hurry the programs for recurrence to the body's hereditary standards.

All the energetic changes effectuated at the magnetic properties field are rapidly and significantly interpreted, as being a part of its own living laws.

As a result of the energetic rectification that appears, a pretty big number of organic chemical compounds, that have become nonspecific, are eliminated from different cellular levels. This generates in the body a quantitative toxicity effect and stages of crisis. Their neutralization is performed at the high specialization level of the hepatic cell, and that's why the hepatic function is very solicited. If this organ is seriously damaged, this will be harder felt. As any disease that has reached a certain degree of evolution weakens the hepatic cell proportionally with its evolution, the cell protection is imposed through interventions with gentle energetic changes to certain intervals, and an alimentary protection during the treatment, too.

The bio-stimulating effect is developed at the cell's level, especially at the level of the cell's membrane. The energetically activity of the cell's membrane increases under the action of the polarized light. The bio-energetically potential of the cell increases. The DNA, the RNA, the protein system and the bio-synthetically processes are activated, determining the increase of the oxygen percentage in blood.

Stimulation means the process of activating the physiological and reconditioning-physiological functions. The electromagnetic field has a direct influence on the nervous-endings and on the nervous system, too. So, the bio-stimulation activates at different levels.

At sub-cellular level, the stereo-chemical changes of the cells accelerate the collagen and albumin synthesis, the oxygen change and the process of the oxide-reducing products (anti-toxic action).

At the cell's level, the potential of the cell's electric field increases, the potential of the cell's membrane changes, the proliferation increases a.s.o.

At the body's level: cell's reorganization, organ's function normalizing, accelerating and maintaining the health condition.

The invention has the following advantages:
the secure obtaining of the necessary frequencies needed in treating some diseases
it doesn't use electromagnetic damaging fields for health
easy to use
it is possible to use the treatment for sick people bearing a pace
marker

1-3. (canceled)

4. A medical apparatus with light flux for biologic treatments, comprising:
an artificial light source and optic filter;
a case;
a support that is set on a foot plate, that bears a light source bulb ventilated by a fan set inside the case;
a concave mirror set on an axial handlebar, on the wall of the case;
an optic filter set on an objective and located on an opposite wall in a slit of the case;
a rotating shutter disc is set on said opposite wall with the optic filter, equipped with a slit regulating device, having orifices c and d that become coaxial on an optic filter axis;
wherein processed light flux is made of rays reflected by the concave mirror and of direct rays reflected by the source of light; and wherein the light flux passes through the orifices c and d of the shutter disc and straighten to the objective, inside of which the optic filter is set.

5. The medical apparatus of claim 4, wherein the rotating speed of the shutter disc is run by an engine, ventilated by a fan, assisted by a digital modulator of the light flux.

6. The medical apparatus of claim 4, wherein a computer controls the entire apparatus' handling and coordinating.

7. The medical apparatus of claim 5, wherein a computer controls the entire apparatus' handling and coordinating.
